(19) United States
(12) Patent Application Publication
(10) Pub. No.: US 2010/0239549 A1
(43) Pub. Date: Sep. 23, 2010

(54) ALPHA LIPOIC ACID CAPSULE COMPOSITION STABILIZED IN WATER PHASE AND METHOD FOR PREPARING THE SAME

(75) Inventors: Chul Hwan Kim, Daejeon (KR); Ki Seok Park, Seongnam (KR); Hyun Nam Yoon, Towaco, NJ (US)

Correspondence Address: HOSOON LEE, 9600 SW OAK ST. SUITE 525, TIGARD, OR, 97223, US

(73) Assignee: DPI SOLUTIONS, INC., Daejeon (KR)

(21) Appl. No.: 12/294,858

(22) PCT Filed: Apr. 26, 2006
(86) PCT No.: PCT/KR2006/001574

§ 371 Date: Sep. 26, 2008

(51) Int. Cl. A61K 8/49; A61Q 19/00

(52) U.S. Cl. 424/94.1; 514/440

(57) ABSTRACT

The present invention relates to an alpha lipoic acid capsule composition stabilized in aqueous phase, which can effectively prevent alpha lipoic acid from decomposing due to external environmental conditions, such as water, temperature, light, and the like, and can maintain the stability of alpha lipoic acid from the interaction between the lipoic acid and other materials, which are added together with the lipoic acid when the lipoic acid is applied to cosmetics, foods, or medical supplies, by mixing water-dispersed alpha lipoic acid, a water-insoluble polymer having self-emulsification properties, and a hydrophilic solvent, thus capsulating alpha lipoic acid, and to a method of preparing the same.

### FIELD OF THE APPLICATION

The present invention relates to an alpha lipoic acid capsule composition stabilized in aqueous phase, which can effectively prevent alpha lipoic acid from decomposing due to external environmental conditions, such as water, temperature, light, and the like, and can maintain the stability of alpha lipoic acid from the interaction between the lipoic acid and other materials, which are added together with the lipoic acid when the lipoic acid is applied to cosmetics, foods, or medical supplies, by mixing water-dispersed alpha lipoic acid, a water-insoluble polymer having self-emulsification properties, and a hydrophilic solvent, thus capsulating alpha lipoic acid, and to a method of preparing the same.

### BACKGROUND OF THE INVENTION

Generally, alpha lipoic acid is known as a material which enhances the immune function of human body, has an excellent antioxidant effect, has excellent blood sugar regulation ability when administered orally, and has very strong antioxidant action. Further, it is known that alpha lipoic acid has functions of removing active oxygen and preventing the formation of wrinkles when it is applied on the skin. Accordingly, alpha lipoic acid has been expected to exhibit excellent functions when it is used for cosmetic compositions, medical compositions, or commodities.

However, alpha lipoic acid has a problem in that, since the alpha lipoic acid has a strong antioxidant function due to the chemical structure of disulfide, the alpha lipoic acid itself is sensitive to external environmental conditions, such as water, temperature, light, and the like, and thus is oxidized and reduced in equilibrium, and particularly, when dithiol is formed through the reduction of the alpha lipoic acid, very bad smells are generated. The reduction reaction of alpha lipoic acid is generally caused by water adjacent to the alpha lipoic acid, and very bad smells are generated even if a small amount of dithiol structure is formed in the reduction reaction. For this reason, although the alpha lipoic acid has very desirable properties, it is extremely limited for a large amount of alpha lipoic acid to be used as an active ingredient in the production of medical supplies, foods, cosmetics and the like.

In order to overcome the problems peculiar to alpha lipoic acid and to improve the stability thereof, International Patent Application Publication No. 2001024795 and U.S. Pat. No. 6,127,394 disclose methods of using amide and ester derivatives of alpha lipoic acid. Further, it is described in those patent documents that these derivatives are decomposed, and thus sustained release occurs. However, once the alpha lipoic acid derivatives are formed, in order to exhibit the efficacy thereof, the structure of the derivatives must be changed into that of the original alpha lipoic acid, even if the derivatives are absorbed in skin through epidermal tissues. Accordingly, there are problems in that the efficiency of alpha lipoic acid is decreased, and the burden on the consumer is increased because the cost of alpha lipoic acid derivatives, prepared through reactions, is very high.

### DISCLOSURE

### Technical Problem

Accordingly, the present invention has been made to overcome the above problems occurring in the prior art, and an object of the present invention is to provide an alpha lipoic acid composition which can be unlimitedly used while maintaining the form of pure alpha lipoic acid, through a method of stabilizing alpha lipoic acid using a micro capsule formed of water-insoluble polymers having self-emulsification properties.

Another object of the present invention is to provide a water-dispersible alpha lipoic acid capsule composition which can be capsulated and can effectively stabilize alpha lipoic acid in an aqueous phase.

A further object of the present invention is to provide a method of preparing a water-dispersed alpha lipoic acid capsule composition which can effectively stabilize alpha lipoic acid in an aqueous phase.

A still further object of the present invention is to provide a cosmetic composition including the water-dispersed alpha lipoic acid capsule composition as an active component.

### Technical Solution

In order to accomplish the above objects, the present inventors completed the present invention, base on the fact that, if alpha lipoic acid and materials added to the alpha lipoic acid to stabilize it are confined in a water-insoluble capsule, the instability of alpha lipoic acid to the external environment, including water, oxygen, heat, air, light, other chemicals, and the like, can be improved.

A water-dispersed alpha lipoic acid capsule composition according to the present invention includes alpha lipoic acid, a water-insoluble polymer having self-emulsification properties or a mixture thereof, an amino acid, and the balance of water, a hydrophilic solvent, or a mixture thereof.

A method of preparing a water-dispersed alpha lipoic acid capsule composition according to the present invention includes a first step of mixing a hydrophilic solvent, alpha lipoic acid, and one or more water-insoluble polymers having self-emulsification properties at room temperature to form a mixture; a second step of adding one or more amino acids to the mixture of the first step to form a mixture; and a third step of preparing a capsule by adding water, a hydrophilic solvent or a mixture thereof to the mixture of the second step.

Another method of preparing a water-dispersed alpha lipoic acid capsule composition includes a first step of mixing a hydrophilic solvent, alpha lipoic acid, and one or more water-insoluble polymers having self-emulsification properties at room temperature to form a mixture; a second step of adding water, a hydrophilic solvent, or a mixture thereof to the mixture of the second step to form a mixture, and then dissolving the mixture; and a third step of mixing one or more amino acids with the mixture of the second step.

Hereinafter, first, a water-dispersed alpha lipoic acid capsule composition of the present invention will be described in detail. The water-dispersed alpha lipoic acid capsule composition of the present invention includes 0.5˜20.0% by weight of alpha lipoic acid, 5.0˜25.0% by weight of one or more water-insoluble polymers having self-emulsification properties, 0.1˜12% by weight of one or more amino acids, and the balance of water, a hydrophilic solvent, or a mixture thereof.

Among the components, the alpha lipoic acid is dissolved in one or more hydrophilic solvents, and is thus maintained in the state of a lipophilic compound, and the alpha lipoic acid is stabilized because it is present in a water-insoluble polymer having self-emulsification properties. In this case, it is preferred that the amount of the alpha lipoic acid be 0.5˜20.0% by weight, based on the total weight of the composition. When the amount thereof is below 0.5% by weight, the amount of alpha lipoic acid that can be stabilized is too little. In contrast, when the amount thereof is above 20.0%, alpha lipoic acid, which is not capsulated, excessively remains in an aqueous phase, and thus there is a problem in that it is difficult to stabilize the alpha lipoic acid.

Meanwhile, the amino acid is formed into a complex together with the alpha lipoic acid which is not capsulated and remains in an aqueous phase. More specifically, the complex is formed between a positive amino acid, which is present in an aqueous phase, and a carboxylic acid of the alpha lipoic acid. The amino acid which can form a complex may include tyrosine, tryptophan, lysine, and the like. It is preferred that the amount of the amino acid that is added be 0.1˜12.0 by weight, and more preferably 0.1˜3.0% by weight, based on the total amount of the composition.

In the present invention, the water-insoluble polymer having self-emulsification properties is not limited, as long as it is not dissolved in water and has self-emulsification properties. More specifically, the water-insoluble polymer used in the present invention includes amphiphilic polymers which can be modified into water-soluble polymers because they react with lipophilic portions of the water-soluble polymers and thus exhibit amphiphilicity, as well as commonly-used water-insoluble polymers having self-emulsification properties, which can be used to capsulate the composition.

The commonly used water-insoluble polymer having self-emulsification properties includes a polystyrene polymer, a polyacryl polymer, a polyolefin polymer, a fluorine polymer, a polyacetal polymer, a polyamide polymer, a polyalkylene polymer, a polyalkenylene polymer, a polyurethane polymer, a polyvinylacetal polymer, a polyvinylbutylal polymer, a polycarbonate polymer, a polyether polymer, a polyester polymer, polyesterurethane polymer, and the like, but is not limited thereto as long as the chemical properties thereof is insolubility.

The amphiphilic polymer includes polyvinyl alcohol ester, polyacrylic acid copolymer, polyacrylic acid ester, polyacrylic acid thioester, vinylpyrrolidone olefin copolymer, polylysine ester, polylysine thioester, hyaluronic acid ester, hyaluronic acid thioester, hyaluronic acid amide, cellulose ester, cyclodextrin ester, gelatin amide, gelatin ester, chitosan amide, and the like.

When the amount of the water-insoluble polymer is small, the amount of alpha lipoic acid that can be capsulated is small, or the alpha lipoic acid cannot be efficiently capsulated. In contrast, when the amount thereof is large, there may be a disadvantage in that the viscosity becomes excessively high. Accordingly, it is preferred that the amount of the water-insoluble polymer having self-emulsification properties or a mixture thereof be 0.5˜25.0% by weight, based on the total weight of the composition.

Consequently, water, a hydrophilic solvent, or a mixture thereof, serving as the material for dissolving the alpha lipoic acid, constitutes the balance of the composition, excluding the alpha lipoic acid and water-insoluble polymer having self-emulsification properties. Any hydrophilic solvent may be used without limitation, as long as it is a polyhydric alcohol. Specifically, the hydrophilic solvent may include ethylene glycol, propylene glycol, diethylene glycol, dipropylene glycol, dibutylene glycol, glycerin, 1,3-butandiol, sorbitol, tetrahydrofuran, and the like. Further, volatile organic solvents, including acetone, ethanol, methanol and the like, may also be used as the hydrophilic solvent together with the polyhydric alcohol, but these volatile organic solvents are not included in a final composition because they are volatilized during the process of preparing a capsule composition.

When a composition including only water as the solvent is used, the usability of the composition is not good. Therefore, generally, a composition including a hydrophilic solvent, such as polyhydric alcohol etc., is used in the related field. Most preferably, the amount of the hydrophilic solvent is 10˜30% by weight, based on the total weight of the composition, but is not limited thereto.

A composition solidified using a spray drying method or a freeze drying method is also included in the water-dispersed alpha lipoic acid composition prepared depending on the above composition. When the solidified composition is prepared, lactic acid, lactose or the like may further be added to the composition in order to easily dry the composition. It is most preferred that the amount thereof be 5˜40% by weight, based on the total weight of the composition.

Further, the present invention provides a cosmetic composition or a food including the water-dispersed alpha lipoic acid capsule composition as an active component. The cosmetic composition or food can be produced using methods commonly used in the related field. Specifically, the cosmetic composition or food can be produced by adding a typical excipient, approved for use in cosmetics, to the water-dispersed alpha lipoic acid capsule composition and then formulating the composition, to which the excipient is added, in dosage forms through commonly used methods. For example, since this composition of the present invention is harmless to the human body, it can be variously applied to an injection, an external preparation, and the like by formulating the composition in the form of a gel, film, bead, mesh, or coated fiber. Further, when the composition is applied on a gauze-shaped non-woven fabric substrate and then dried, a cosmetic material having effects such as whitening ability, the removal of dead skin, and the like can be produced. A general cosmetic composition may include 0.001˜5.0% by weight of the alpha lipoic acid of the present invention as an active component.

Hereinafter, a method of preparing a water-dispersed alpha lipoic acid capsule composition will be described in more detail.

The method of preparing a water-dispersed alpha lipoic acid capsule composition according to the present invention includes a first step of mixing a hydrophilic solvent, alpha lipoic acid, and one or more water-insoluble polymers having self-emulsification properties at room temperature to form a mixture; a second step of adding one or more amino acids to the mixture of the first step to form a mixture; and a third step of preparing a capsule by adding water, a hydrophilic solvent or a mixture thereof to the mixture of the second step.

Further, in the present invention, the composition may be prepared by simultaneously adding and mixing the components, but may be prepared by sequentially adding and mixing the components. Accordingly, the method of preparing a water-dispersed alpha lipoic acid capsule composition may include a first step of mixing a hydrophilic solvent, alpha lipoic acid, and one or more water-insoluble polymers having self-emulsification properties at room temperature to form a mixture; a second step of preparing a capsule by adding water, a hydrophilic solvent or a mixture thereof to the mixture of the first step; and a third step of adding one or more amino acids to water after the second step of preparing a capsule.

A polyhydric alcohol or a mixture of a volatile organic solvent and the polyhydric alcohol may be used as the hydrophilic solvent. However, the volatile organic solvent is completely removed after the step of preparing a capsule, and thus only the polyhydric alcohol remains as the hydrophilic solvent, thereby constituting a capsule composition.

The water-dispersed alpha lipoic acid of the present invention, prepared through these methods, is capsulated in a polymer having self-emulsification properties, so that the water-insoluble polymer having self-emulsification properties forms a matrix, with the result that complexes of alpha lipoic acid are dispersed in the matrix of the water-insoluble polymer having self-emulsification properties. When a mixture of the water-insoluble polymer having self-emulsification properties and complexes of alpha lipoic acid is dispersed in water while being stirred, the water-insoluble polymer having self-emulsification properties forming the matrix is precipitated. In this case, the alpha lipoic acid is capsulated while the precipitated matrix surrounds the dispersed mixture of the water-insoluble polymer having self-emulsification properties and complexes of alpha lipoic acid. The alpha lipoic acid capsule composition prepared through this capsulation process can effectively prevent alpha lipoic acid from decomposing due to external environmental conditions, such as water, temperature, light, and the like, and can also maintain the stability of alpha lipoic acid under the interaction between the lipoic acid and other materials, which are added together with the lipoic acid when the lipoic acid is applied to cosmetics.

Particularly, in the alpha lipoic acid capsule, prepared by capsulating the alpha lipoic acid in the water-insoluble polymer having self-emulsification properties and dispersing the capsulated alpha lipoic acid in water containing amino acids, the alpha lipoic acid is capsulated by the water-insoluble polymer having self-emulsification properties in a solid phase, and can thus be stably maintained in the presence of external stimuli such as oxygen, water, light and the like, and part of the alpha lipoic acid can be stabilized in a aqueous phase when the part of alpha lipoic acid included in the solid phase is eluted from water.

### DESCRIPTION OF DRAWINGS

FIG. 1 is a photograph showing an alpha lipoic acid composition using an electron scanning microscope, according to the present invention.

### MODE FOR INVENTION

Hereinafter, the present invention will be described in more detail with reference to preferred Examples and Comparative Examples.

TABLE 1
Examples 1~18 and Comparative Examples 1, 2

| | | Example | | | | | | | | |
|---|---|---|---|---|---|---|---|---|---|---|
| | | 1 | 2 | 3 | 4 | 5 | 6 | 7 | 8 | 9 |
| Composition | | 10 | 11 | 12 | 13 | 14 | 15 | 16 | 17 | 18 |
| Hydrophilic solvent | Glycerin | 10 | — | — | — | — | — | — | — | — |
| | Diethylene glycol | — | 10 | — | 10 | 10 | 30 | 30 | 30 | 30 |
| | Dipropylene glycol | — | — | 10 | — | — | — | — | — | — |
| | Ethanol | — | — | — | 20 | 20 | — | — | — | — |
| | Acetone | 50 | 50 | 50 | 30 | 30 | 30 | 30 | 30 | 30 |
| | Methanol | — | — | — | — | — | 20 | 20 | 20 | 20 |
| Amino acid | Tyrosine | 2 | — | 1 | — | 1.5 | — | 0.5 | 3 | — |
| | Lysine | — | 2 | — | 1 | — | 0.5 | — | — | 3 |
| Alpha lipoic acid | | 5 | 20 | 15 | 5 | 10 | 15 | 5 | 10 | 15 |
| Water-insoluble polymer | Polyester ionomer | 10 | 20 | — | — | — | — | — | — | 15 |
| having self-emulsification | Polystyrene ionomer | — | — | 8 | 15 | — | — | 10 | 5 | — |
| properties | | | | | | | | | | |

| | | | | | | | | | |
|---|---|---|---|---|---|---|---|---|---|
| Water | to 100 | to 100 | to 100 | to 100 | to 100 | to 100 | to 100 | to 100 | to 100 |

### Method of Preparing Water-Dispersed Alpha Lipoic Acid Capsule Composition

### Examples 1˜9

i) one or more hydrophilic solvents were put in a flask at room temperature, and alpha lipoic acid was added thereto, as set forth in Table 1, and was then dissolved in the mixture.

ii) a water-insoluble polymer having self-emulsification properties was dissolved in a hydrophilic solvent. If necessary, the process of dissolving the water-insoluble polymer having self-emulsification properties can be performed while heating it. After the dissolution process was completed, the solution was cooled to room temperature again.

iii) the solution formed in the step i) was mixed with the solution of the aqueous water-insoluble polymer having self-emulsification properties formed in the step ii).

iv) the mixed solution formed in the step iii) was mixed with a mixture of water and a hydrophilic solvent while slowly stirring it, and thus a capsule was prepared.

v) after the capsule was completely prepared, solvents, which are volatilized at a low pressure of 100 mHg and a temperature of 60° C., were removed from the capsule. Among the organic solvents used in the Examples, acetone, ethanol and methanol were easily removed.

vi) after all of the above processes were performed, amino acids were added to the water-phased capsule as given in Table 1.

### Examples 10˜18

i) first, one or more hydrophilic solvents were put in a flask, and alpha lipoic acid was added thereto, as set forth in Table 1, and was then dissolved in the mixture.

ii) amino acids were dispersed or dissolved in the solution formed in the step i).

iii) a water-insoluble polymer having self-emulsification properties was dissolved in a hydrophilic solvent. If necessary, the process of dissolving the water-insoluble polymer having self-emulsification properties can be performed while heating it. After the dissolution process was completed, the solution was cooled to room temperature again.

iv) the solution formed in the step ii) was mixed with the solution of the aqueous water-insoluble polymer having self-emulsification properties formed in the step iii).

v) the mixed solution formed in the step iv) was mixed with a mixture of water and a hydrophilic solvent while slowly stirring it, and thus a capsule was prepared.

vi) after the capsule was completely prepared, solvents, which are volatilized at a low pressure of 100 mHg and a temperature of 60° C., were removed from the capsule. Among the organic solvents used in the Examples, acetone, ethanol and methanol were easily removed.

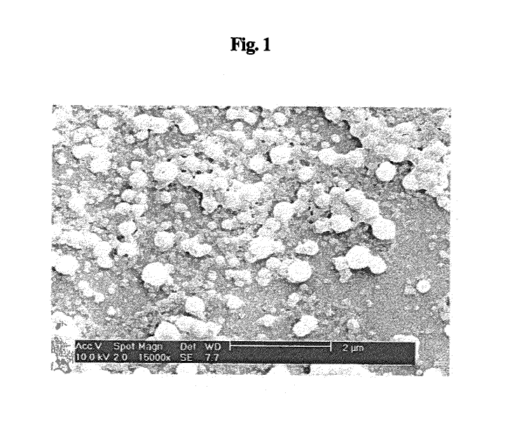

FIG. 1 is a photograph showing the capsule composition of Example 1 prepared through the above method using an electron scanning microscope. In FIG. 1, it can be seen that the composition of the present invention was prepared in the form of capsule.

### Comparative Example 1

### Titer Determination of Non-Capsulated Alpha Lipoic Acid

In Comparative Example 1, an alpha lipoic acid composition was prepared through the same process of adding other components as in Example 1, except for the process of capsulating the alpha lipoic acid using polyester ionomer, which is a water-insoluble polymer having self-emulsification properties.

### Comparative Example 2

### Titer Determination of Alpha Lipoic Acid Including No Amino Acids

In Comparative Example 2, an alpha lipoic acid composition was prepared through the same process as in Example 1, except for the process of adding amino acids.

### Experimental Example 1

### Titer Determination of Alpha Lipoic Acid

The initial titer of alpha lipoic acid in the composition of each of the Examples 1˜9 and Comparative Examples 1˜2 was set to 100. After one month, titers of alpha lipoic acids were respectively determined at room temperature, 37° C. and 45° C., and the results thereof are given in Table 2. The titer thereof was determined using an HP1090 HPLC system, available from Hewlett-Packard Development Company, L.P., DAD 280 nm was used as a detector, and Agilent XDB C-18 was used as a column. Before the titer determination, samples were used after the water-insoluble capsule was collapsed from the samples and the samples were pretreated, that is, diluted about 100 times using acetonitrile.

TABLE 2

| | | | | | | | | | | Com- | |
|---|---|---|---|---|---|---|---|---|---|---|---|
| | | | | | | | | | | parative | |
| | Example | | | | | | | | | Example | |
| | 1 | 2 | 3 | 4 | 5 | 6 | 7 | 8 | 9 | 1 | 2 |
| Room | 100 | 99 | 100 | 100 | 100 | 99 | 99 | 99 | 98 | 75 | 95 |
| tem- | | | | | | | | | | | |
| pera- | | | | | | | | | | | |
| ture | | | | | | | | | | | |
| 37° C. | 100 | 99 | 99 | 98 | 99 | 99 | 99 | 97 | 97 | 65 | 92 |
| 45° C. | 99 | 98 | 99 | 97 | 99 | 98 | 97 | 97 | 95 | 57 | 87 |

As shown in Table 2, the titers of the compositions of Comparative Example 1, in which only amino acids were added, without adding a water-insoluble polymer having self-emulsification properties, and Comparative Example 2, in which only a water-insoluble polymer having self-emulsification properties was added, and thus alpha lipoic acid was capsulated without adding amino acids, were very low at room temperature as well as at high temperature, compared to these of the compositions of Examples. Accordingly, it can be seen that the compositions of Examples have much higher stability than these of Comparative Examples 1 and 2.

### Example 19

### Preparation of Cosmetic Composition Including Alpha Lipoic Capsule

A cream base was obtained from Amorepacific Corporation and used in the preparation of a cosmetic composition. The cosmetic composition including alpha lipoic acid was prepared by mixing 90% of a cream composition with 10% of the capsule composition prepared in Example 1. Each of the prepared cosmetic compositions was stored at a temperature of 35° and at room temperature for two months, and then the smells thereof were observed using a sensory test. At this time, it was found that no alpha lipoic acid smell was given off.

As described above, although the preferred embodiments of the present invention have been disclosed for illustrative purposes, those skilled in the art will appreciate that various modifications, additions and substitutions are possible, without departing from the scope and spirit of the invention as disclosed in the accompanying claims.

### INDUSTRIAL APPLICABILITY

According to the present invention, there can be provided a water-dispersed alpha lipoic acid capsule composition formed in a nanoparticle structure, which can effectively prevent alpha lipoic acid from decomposing due to external environmental conditions, such as water, temperature, light, and the like, and can maintain the stability of alpha lipoic acid under the interaction between the lipoic acid and other materials, which are added together with the lipoic acid when the lipoic acid is applied to cosmetics, and thus can be usefully used for cosmetic compositions, medical compositions, food, and the like.

1. A water-dispersed alpha lipoic acid capsule composition, comprising:
0.5-20.0% by weight of alpha lipoic add;
0.1-12% by weight of one or more amino acids;
5.0-25.0% by weight of one or more water-insoluble polymers having self-emulsification properties; and
a balance of water, a hydrophilic solvent, or a mixture thereof.

2. The water-dispersed alpha lipoic acid capsule composition according to claim 1, wherein an amount of the hydrophilic solvent is 10-30% by weight.

3. The water-dispersed alpha lipoic acid capsule composition according to claim 1, wherein the water-insoluble polymer having self-emulsification properties is one or more selected from the group consisting of a polystyrene polymer, a polyacryl polymer, a polyolefin polymer, a fluorine polymer, a polyacetal polymer, a polyamide polymer, a polyalkylene polymer, a polyalkenylene polymer, a polyurethane polymer, a polyvinylacetal polymer, a polyvinylbutylal polymer, a polycarbonate polymer, a polyether polymer, a polyester polymer, and polyesterurethane polymer.

4. The water-dispersed alpha lipoic acid capsule composition according to claim 1, wherein the water-insoluble polymer having self-emulsification properties is one or more selected from the group consisting of polyvinyl alcohol ester, polyacrylic copolymer, polyacrylic acid ester, polyacrylic add thioester, vinylpyrrolidone olefin copolymer, polylysine ester, polylysine adoester, hyaluronic acid ester, hyaluronic acid thioester, hyaluronic acid amide, cellulose ester, cyclodextrin ester, gelatin amide, gelatin ester, chitosan amide, and a substance for forming a liposome.

5. The water-dispersed alpha lipoic acid capsule composition according to claim 1, wherein the hydrophilic solvent is a polyhydric alcohol

6. The water-dispersed alpha lipoic acid capsule composition according to claim 5, wherein the polyhydric alcohol is one or more selected from the group consisting of ethylene glycol, propylene glycol, diethylene glycol, dipropylene glycol, dibutylene glycol, glycerin, 1,3-butandiol, sorbitol, and tetrahydrofuran.

7. The water-dispersed alpha lipoic acid capsule composition according to claim 1, wherein an amount of the amino acid is 0.1-3.0% by weight.

8. The water-dispersed alpha lipoic acid capsule composition according to claim 1, wherein the alpha lipoic acid composition is solidified using a spray drying method ox a freeze drying method.

9. A method of preparing a water-dispersed alpha lipoic acid capsule composition, comprising:
a) a first step of mixing a hydrophilic solvent, alpha lipoic acid, and one or more water-insoluble polymers having self-emulsification properties at room temperature to form a mixture;
b) a second step of adding one or more amino acids to the mixture of the first step to form a mixture; and
c) a third step of preparing a capsule by adding water, a hydrophilic solvent or a mixture thereof to the mixture of the second step.

10. The method of preparing a water-dispersed alpha lipoic acid capsule composition according to claim 9, wherein the hydrophilic solvent is a mixture of a volatile organic solvent and a polyhydric alcohol, and the volatile organic solvent is removed after the step of preparing a capsule.

11. A method of preparing a water-dispersed alpha lipoic acid capsule composition, comprising:
a) a, first step of mixing a hydrophilic solvent, alpha lipoic acid, and one or more water-insoluble polymers having self-emulsification properties at room temperature to form a mixture;
b) a second step of adding water, a hydrophilic solvent or a mixture thereof to the mixture of the second step to form a mixture and then dissolving the mixture; and
c) a third step of mixing one or more amino acids with the mixture of the second step.

12. A cosmetic composition, comprising the water-dispersed alpha lipoic acid capsule composition according to claim 1 as an active component.

13. A cosmetic composition according to claim 12, wherein an amount of the water-dispersed alpha lipoic acid capsule composition is 0.001-5.0% by weight.
